(19) United States
(12) Patent Application Publication
(10) Pub. No.: US 2020/0275873 A1
(43) Pub. Date: Sep. 3, 2020

(54) EMOTION ANALYSIS METHOD AND DEVICE AND COMPUTER READABLE STORAGE MEDIUM

(71) Applicant: BOE Technology Group Co., Ltd., Beijing (CN)

(72) Inventor: Zhihong Xu, Beijing (CN)

(21) Appl. No.: 16/547,790

(22) Filed: Aug. 22, 2019

(30) Foreign Application Priority Data
Feb. 28, 2019 (CN) 201910151951.1

(51) Int. Cl. A61B 5/16; G06K 9/00; G10L 25/63; A61B 5/1171; A61B 5/1172; A61B 5/0402; A61B 5/0476; A61B 5/0496; A61B 5/00

(52) U.S. Cl. CPC A61B 5/165; G06K 9/00302; G06K 9/00275; G06K 9/00335; G06K 9/00087; A61B 5/4803; A61B 5/1176; A61B 5/1172; A61B 5/0402; A61B 5/0476; A61B 5/0496; G10L 25/63

(57) ABSTRACT

The embodiments of the present disclosure provide an emotion analysis method, an emotion analysis device, and a computer readable medium. The motion analysis method includes: collecting a facial image and body parameter information of a target object; and recognizing an expression of the target object according to the facial image, and determining an state of the target object according to the recognized expression in combination with the body parameter information.

### CROSS-REFERENCE TO RELATED APPLICATION(S)

This application claims priority to the Chinese Patent Application No. 201910151951.1, filed on Feb. 28, 2019, which is incorporated herein by reference in its entirety.

### TECHNICAL FIELD

The present disclosure generally relates to the field of artificial intelligence, and more particularly, to an emotion analysis method, an emotion analysis device, and a computer readable medium.

### BACKGROUND

Human emotions play an important role in people's social interactions, and emotion analysis techniques are receiving more and more attention in the field of artificial intelligence.

Conventional emotion analysis methods usually recognize an emotion based on a person's facial expression or voice. But a human emotion is a result characterized by comprehensive factors. If the human emotion is determined only by an expression or voice, the determination has low accuracy, and it is impossible to provide good user experience.

### SUMMARY

According to an aspect of the embodiments of the present disclosure, there is provided an emotion analysis method, comprising:

collecting a facial image and body parameter information of a target object; and

recognizing an expression of the target object according to the facial image, and determining an emotional state of the target object according to the recognized expression in combination with the body parameter information.

In an example, the body parameter information comprises acceleration information of the target object.

In an example, recognizing an expression of the target object according to the facial image comprises:

applying a deep convolutional neural network algorithm to the facial image to obtain an emotion recognition feature vector; and

applying a Support Vector Machine (SVM) algorithm to the obtained emotion recognition feature vector to determine the expression of the target object.

In an example, the expression comprises one of neuter, happiness, surprise, sadness, anger, disgust, fear or contempt.

In an example, determining an emotional state of the target object according to the recognized expression in combination with the body parameter information comprises:

comparing the acceleration information with reference information;

determining a behavioral performance of the target object according to a comparison result; and

determining a real-time emotional state of the target object by combining the determined expression with the determined behavioral performance.

In an example, the behavioral performance comprises one of ‘tension’, “calmness” or “negativity”.

In an example, the emotion analysis method further comprises:

determining whether the target object has an abnormal emotion according to the obtained emotional state, and if so, issuing a reminder.

In an example, the emotion analysis method further comprises: collecting fingerprint information of the target object, and recognizing an identity of the target object according to the collected facial image or fingerprint information.

In an example, the body parameter information comprises physiological information of the target object.

In an example, the physiological information comprises: electrocardiogram information, electroencephalogram information, electrooculogram information, and voice information.

In an example, recognizing an expression of the target object according to the facial image, and determining an emotional state of the target object according to the recognized expression in combination with the body parameter information comprises:

performing electrocardiogram analysis based on the electrocardiogram information, performing electroencephalogram analysis based on the electroencephalogram information, performing electrooculogram analysis based on the electrooculogram information, and determining a first emotional stress of the target object based on analysis results of the electrocardiogram analysis, the electroencephalogram analysis and the electrooculogram analysis;

performing voice analysis based on the voice information to determine a second emotional stress of the target object;

performing facial expression analysis based on the facial image to determine a third emotional stress of the target object; and

performing comprehensive analysis on the first emotional stress, the second emotional stress, and the third emotional stress to determine an emotional stress state of the target object.

In an example, determining a first emotional stress of the target object based on analysis results of the electrocardiogram analysis, the electroencephalogram analysis and the electrooculogram analysis comprises:

applying a Decision Tables and Naive Bayes (DTNB) algorithm using at least one of a low frequency power LF, a high frequency power HF, and a high frequency power to low frequency power ratio HF/LF of heart rate variability obtained by the electrocardiogram analysis, at least one of an alpha rhythm, a Beta rhythm, and an ApEn+LLE feature obtained by the electroencephalogram analysis, and an electrooculographic behavior trajectory determined by the electrooculogram analysis as inputs to obtain the first emotional stress of the target object.

In an example, the facial expression analysis comprises face detection, face recognition, and emotion recognition.

In an example, performing comprehensive analysis on the first emotional stress, the second emotional stress, and the third emotional stress to determine the emotional stress state of the target object comprises: inputting the first emotional stress, the second emotional stress, and the third emotional stress to a Bayesian network to obtain a comprehensive evaluation result as the emotional stress state of the target object.

In an example, the emotion analysis method further comprises: recognizing an identity of the target object according to the collected facial image.

In an example, the emotion analysis method further comprises: presenting the emotional state of the target object.

According to another aspect of the embodiments of the present disclosure, there is provided an emotion analysis device, comprising a memory and a processor, the memory having stored thereon instructions which, when executed by the processor, cause the processor to perform the emotion analysis method described above.

According to yet another aspect of the embodiments of the present disclosure, there is provided a computer readable storage medium having stored thereon computer readable instructions which, when executed by a computer, cause the computer to perform the emotion analysis method described above.

### BRIEF DESCRIPTION OF THE ACCOMPANYING DRAWINGS

The above and other purposes, features and advantages of the present disclosure will become more understandable from the following description of the embodiments of the present disclosure in conjunction with the accompanying drawings. Components in the accompanying drawings are only for the purpose of illustrating the principles of the present disclosure. In the accompanying drawings, the same or similar technical features or components will be denoted by the same or similar reference signs.

FIG. 1 illustrates a flowchart of an emotion analysis method according to an embodiment of the present disclosure;

FIG. 2 illustrates a schematic block diagram of an emotional analysis device according to an embodiment of the present disclosure;

FIG. 3 illustrates a flowchart of an emotion analysis method according to an embodiment of the present disclosure;

FIG. 4a illustrates a flowchart of an example of step S202 of FIG. 3;

FIG. 4b illustrates a flowchart of an example of step S203 of FIG. 3;

FIG. 5 illustrates a schematic block diagram of an emotion analysis device according to another embodiment of the present disclosure;

FIG. 6 illustrates a schematic block diagram of a multi-dimensional information collection module of FIG. 5;

FIG. 7 illustrates a flowchart of an emotion analysis method according to another embodiment of the present disclosure;

FIG. 8 illustrates a flowchart of an example of step S302 of FIG. 7; and

FIG. 9 illustrates a schematic diagram of a computer device according to an embodiment of the present disclosure.

### DETAILED DESCRIPTION

Embodiments of the present disclosure will be described below with reference to the accompanying drawings. Elements and features described in one figure or one embodiment of the present disclosure may be combined with elements and features illustrated in one or more other figures or embodiments. It should be illustrated that, for the sake of clarity, representations and descriptions of components and processes which are not known to those of ordinary skill in the art are omitted in the accompanying drawings and the description.

The embodiments of the present disclosure propose an emotion analysis method and device, in which a real-time emotional state of a target object is analyzed by combining expression information obtained by face recognition analysis with body parameter information, which may more accurately determine the real-time emotional state of the target object, and is convenient for reminding the target object to adjust his/her emotion in time and maintain a healthy state of mind.

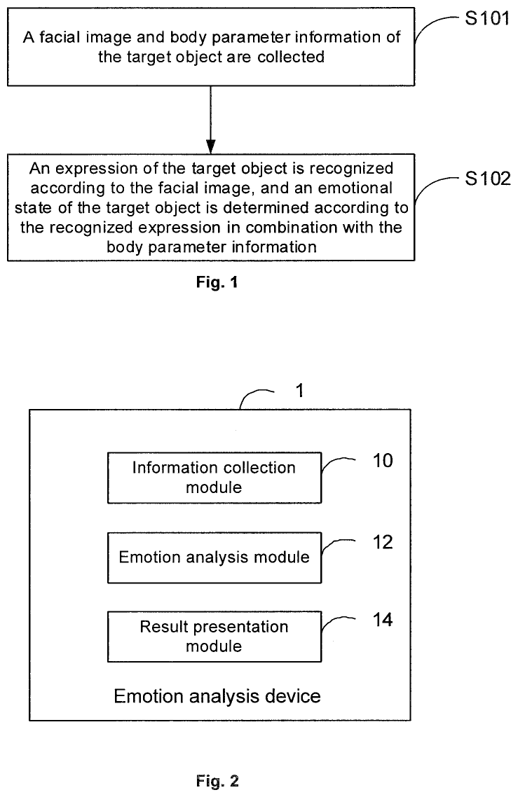

FIG. 1 illustrates a flowchart of an emotion analysis method according to an embodiment of the present disclosure.

In step S101, a facial image and body parameter information of the target object are collected. In some embodiments, the body parameter information may comprise acceleration information of the target object. In some other embodiments, the body parameter information may comprise physiological information of the target object.

In step S102, an expression of the target object is recognized according to the facial image, and an emotional state of the target object is determined according to the recognized expression in combination with the body parameter information.

In a case where the body parameter information comprises the acceleration information of the target object, the expression of the target object may be determined by using a deep convolutional neural network and applying a Support Vector Machine (SVM), a behavioral performance of the target object is determined based on the acceleration information, and the real-time emotional state of the target object is determined by combining the expression with the behavioral performance.

In a case where the body parameter information comprises the physiological information of the target object, an emotional stress of the target object in expression may be obtained by analyzing a facial expression, an emotional stress of the target object in physiology may be obtained by analyzing the physiological information, and the emotional stress state of the target object is obtained by comprehensively analyzing both of the emotional stresses through a Bayesian network.

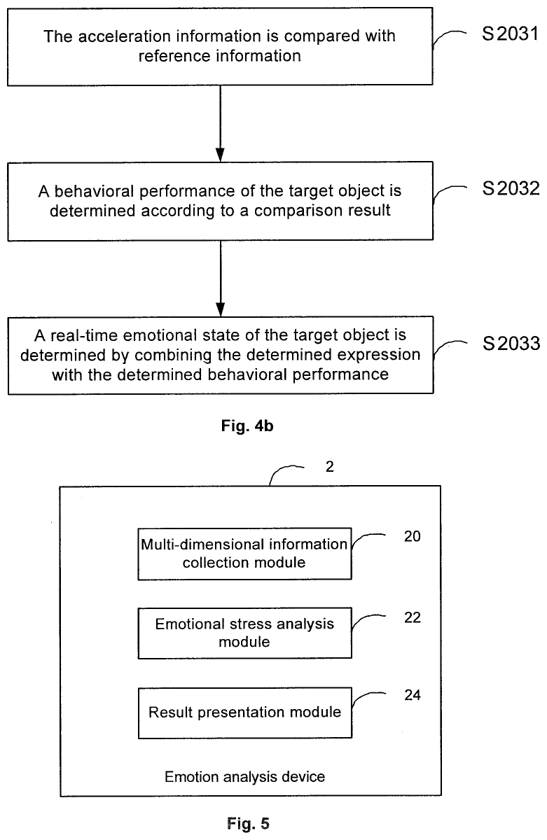

An embodiment of the emotion analysis device and method in a case where the body parameter information comprises the acceleration information of the target object will be described below with reference to FIGS. 2, 3, and 4a and 4b (hereinafter collectively referred to as FIG. 4).

On the other hand, the present disclosure proposes an emotion analysis device. FIG. 2 illustrates a schematic block diagram of an emotional analysis device according to an embodiment of the present disclosure. In the present embodiment, the body parameter information comprises acceleration information of the target object. As shown in FIG. 2, the emotion analysis device 1 comprises: an information collection module 10 configured to collect a facial image or fingerprint information and acceleration information of a target object; an emotion analysis module 12 configured to recognize an expression and an identity of the target object according to the facial image or the fingerprint information of the target object, and analyze a real-time emotional state of the target object by combining the expression information with the acceleration information; and a result presentation module 14 configured to present the emotional state of the target object.

In the emotion analysis device according to the embodiment of the present disclosure, the real-time emotional state of the target object is comprehensively analyzed by combining the expression information obtained by the face recognition analysis with the acceleration information, which may more accurately reflect a person's real-time emotional state, and is convenient for reminding the target object to adjust his/her emotion in time and maintain a healthy state of mind.

According to a specific embodiment of the present disclosure, the information collection module 10 may comprise an image collection module, a fingerprint information collection module, and an acceleration information collection module. The image collection module may be implemented with a variety of cameras.

The person's identity may be recognized using a software program or algorithm based on the collected facial image. For example, a process of recognizing the person's identity based on a combination of a second generation resident Identity (ID) card and a face recognition algorithm is as follows: automatically collecting information of an ID card presented by a user using a second generation resident ID card reader, collecting a facial image of the user through a high-definition camera, and finally determining a face similarity between an avatar photo of the ID card and the image collected by the camera based on the avatar photo and the image using a depth learning algorithm.

The person's identity may be recognized based on image recognition, or may also be recognized based on fingerprint information collected by a fingerprint sensor. For example, examples of the fingerprint sensor comprise, but not limited to, an optical fingerprint sensor, a semiconductor capacitance sensor, a semiconductor thermal sensor, a semiconductor pressure sensor, an ultrasonic sensor, and a Radio Frequency (RF) sensor. In a case of an optical fingerprint sensor, a variation relationship of a geometric parameter such as translation, rotation etc. which may exist between two fingerprints may be searched based on related objective function and genetic operators, a matching relationship between data in a fingerprint database and a fingerprint which is collected in real time is determined based thereon, to determine a degree of matching between the two fingerprints, and an identity of the target object is determined by creating a fingerprint database or referring to an existing fingerprint database and searching the fingerprint database for fingerprint information.

The acceleration information collection module may monitor the acceleration information of the target object. For example, the acceleration information collection module may comprise an accelerometer to collect the acceleration information of the target object. The acceleration information may be gait acceleration information. For example, when an absolute value of gait acceleration is less than or equal to 0.2 kg/m³, it indicates that a measured person's emotion is gentle (i.e., his/her behavioral performance is “calmness”); when a value of the gait acceleration is negative, and the absolute value of the gait acceleration is greater than 0.2 kg/m³, it indicates that the measured person's emotion is depressed (i.e., his/her behavioral performance is “negativity”); and when the gait acceleration is positive, and the absolute value of the gait acceleration is greater than 0.2 kg/m³, it indicates that the measured person's emotion is happy (i.e., his/her behavioral performance is “tension”). The acceleration information collection module may further comprise acceleration information obtained from other motion of the monitored person or from a health monitoring device. The acceleration information collection module comprises an acceleration analysis module configured to perform analysis based on the monitored acceleration information of the monitored person. For example, according to a rule of acceleration research results described above, the behavioral performance of the target object may be classified as “tension”, “calmness” or “negativity”.

The person's expression may be recognized by analyzing the facial image. An emotion recognition feature vector may be acquired based on a deep Convolutional Neural Network (CNN) algorithm, and then a real-time emotional state of the target object may be analyzed. A general process of acquiring an emotion recognition feature vector using the deep convolutional neural network is as follows.

In step 1, a camera and a collection program are started.

In step 2, a facial image is collected using the camera.

In step 3, face detection is performed using a face detection algorithm.

In step 4, a deep convolutional neural network model is constructed.

In step 5, a facial image which is detected in real time is input.

In step 6, a real-time emotional state output value is obtained.

The emotional state output value represents the emotion recognition feature vector.

According to the method of the embodiment described above, more useful features may be learned by constructing a machine learning model with multiple hidden layers and a large amount of training data, thereby finally improving the accuracy of emotion classification or prediction.

According to a specific embodiment of the present disclosure, after the emotion analysis module 12 acquires the emotion recognition feature vector, the emotion analysis module 12 may obtain an emotion recognition result based on the SVM algorithm. A process of recognizing an emotion based on the SVM algorithm is as follows.

In step 1, an SVM algorithm model is constructed.

In step 2, a real-time emotional state value is input.

In step 3, an emotion recognition result is output. For example, the expression (emotion) of the target object may be determined as one of neuter, happiness, surprise, sadness, anger, disgust, fear or contempt.

The SVM network model may be fixed or parameters of the SVM network model may be transformed according to actual conditions.

In some embodiments, the real-time emotional state of the target object may be obtained by combining the behavioral performance (for example, “tension”, “calmness”, or “negativity”) determined based on the acceleration information with the expression (for example, one of neuter, happiness, surprise, sadness, anger, disgust, fear or contempt) determined based on the facial image. For example, the behavioral performance of “calmness” and the expression of “happiness” may be combined into a real-time emotional state of “calm happiness”. Of course, the embodiments of the present disclosure are not limited thereto, and any other combination manner may be used as needed to obtain the real-time emotional state of the target object.

According to the method of the embodiment described above, the problem of deep learning may be solved with a small number of samples, and therefore the emotion recognition result has a certain generalization ability.

According to the method of the embodiment described above, the real-time emotional state of the target object is determined by combining the expression information with the acceleration information, which not only simplifies the emotional classification type of the target object, but also accurately determines emotional features of the target object.

According to a specific embodiment of the present disclosure, the emotion analysis device further comprises a result presentation module 14 which may present and report emotional health state data of the target object in real time. The report may be implemented in a variety of forms, such as voice, image, text message prompt etc. This facilitates timely discovery of the emotional state of the monitored person. By performing statistics on emotional health data accumulated over time, a rule according to which an emotion of the monitored person occurs may further be discovered, and a countermeasure is formulated, which improves the mental health or personal mental state level. This data may be used by the monitored person for reference or may be used by a doctor or a family member for reference in a case of medical care.

According to a specific embodiment of the present disclosure, the result presentation module 14 comprises a reminder module configured to issue a reminder to the target object or a monitoring person when it is determined that the target object has an abnormal emotion. This is beneficial for reminding the monitored person to adjust himself/herself in time, or reminding the monitoring person, such as the doctor or the family member, to prevent accidents.

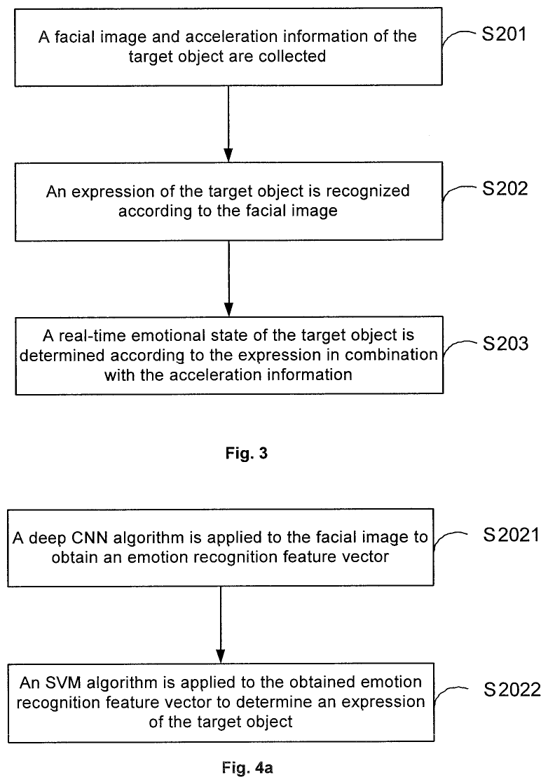

The present disclosure further provides an emotion analysis method, which will be described below with reference to FIG. 3. FIG. 3 illustrates a flowchart of an emotion analysis method according to an embodiment of the present disclosure. In the present embodiment, the body parameter information comprises acceleration information of the target object. As shown in FIG. 3, the emotion analysis method comprises the following steps.

In step S201, a facial image and acceleration information of the target object are collected.

In step S202, an expression of the target object is recognized according to the facial image.

In step S203, a real-time emotional state of the target object is determined according to the expression in combination with the acceleration information.

In some embodiments, the emotion analysis method further comprises: acquiring an emotion recognition feature vector based on a deep CNN algorithm when the real-time emotional state of the target object is analyzed, thereby analyzing the real-time emotional state of the target object. According to a specific embodiment of the present disclosure, the emotional health analysis method further comprises: obtaining an emotion recognition result based on the SVM algorithm after acquiring the emotion recognition feature vector.

In some embodiments, it may also be determined whether the target object has an abnormal emotion according to the obtained emotional state, and if so, a reminder that the emotion is abnormal is issued.

In some embodiments, the emotion analysis method may further comprise: collecting fingerprint information of the target object, and recognizing an identity of the target object according to the collected facial image or fingerprint information. The operation may be performed at any desired time as needed, for example, the step of collecting the target image may be performed together with the collection of the facial image in step S201, and the step of recognizing the identity may be performed at any desired time after step S201, which will not be described in detail here.

In some embodiments, the emotion analysis method may further comprise: presenting the emotional state of the target object.

FIG. 4a illustrates a flowchart of an example of step S202 of FIG. 3.

In step S2021, a deep CNN algorithm is applied to the facial image to obtain an emotion recognition feature vector.

As an example, as shown in Table 1 below, a deep CNN structure comprising twelve convolutional layers, four pooling layers, and two fully connected layers ma be used.

| Input | Name |
|---|---|
| Cov5-16 | C1 |
| Cov5-16 | C2 |
| Max pooling | S1 |
| Cov5-16 | C3 |
| Cov5-16 | C4 |
| Max pooling | S2 |
| Cov3-32 | C5 |
| Cov3-32 | C6 |
| Cov3-32 | C7 |
| Cov3-32 | C8 |
| Max pooling | S3 |
| Cov3-32 | C9 |
| Cov3-32 | C10 |
| Cov3-32 | C11 |
| Cov3-32 | C12 |
| Max pooling | S4 |
| Full connection-200 | FC-200 |
| SVM | SVM |

C1, C2, C3, and C4 are all convolutional layers. Each layer uses sixteen convolution kernels, each of which has a size of 5*5. C5, C6, C7, C8, C9, C10, C11, and C12 are all convolutional layers. Each layer uses thirty-two convolution kernels, each of which has a size of 3*3. S1, S2, S3, and S4 are all pooling layers and are all maximum pooling layers. The FC-200 layer is a fully connected layer which generates a 200-dimensional vector. The ELU function is selected as an activation function, and a linear learning rate attenuation method is used for expression recognition.

In step 1, an emotional feature vector X is input.

In step 2, for the feature vector X, a convolutional layer uses a 5*5 convolution window for feature sampling; and an output value X₁ of the convolutional layer is obtained after processing X using the following formula,

X₁=Activity(Wₖ⁽¹⁾*X+bₖ⁽¹⁾)

where Activity( ) represents an activation function, Wₖ⁽¹⁾ represents a weight vector of a neuron, a uniform distribution is applied to Wₖ⁽¹⁾ to obtain an initialization vector, superscripts 1 of W and b represent a first layer, and subscripts k of W and b represent a k^{th} parameter of the layer.

As an example, the following function ELU( ) is selected as the activation function.

$$E L U ( x ) = \{ \begin{matrix}x , x > 0 \\ α ( {e}^{x} - 1 ) , x \leq 0\end{matrix}$$

Update of weight vectors of a part of neurons with a weight vector of Wₖ⁽¹⁾ is stopped by a Dropout method and a uniform distribution is applied thereto to obtain an initialization vector.

In step 3, for the feature vector X₁, a convolutional layer uses a 5*5 convolution kernel size, and an output value X₂ of the convolutional layer is obtained after processing X₁ using the following formula.

X₂=Activity(Wₖ⁽²⁾*X₁+bₖ⁽²⁾)

where a weight vector of a neuron is Wₖ⁽²⁾, a uniform distribution is applied to Wₖ⁽²⁾ to obtain an initialization vector, a function ELU( ) is used as an activation function, update of weight vectors of a part of neurons with a weight vector of Wₖ⁽²⁾ is stopped by a Dropout method and a uniform distribution is applied thereto to obtain an initialization vector Wₖ⁽³⁾.

X₂=Activity(Wₖ⁽²⁾*X₁+bₖ⁽²⁾)

In step 4, a new feature value X₃ is re-extracted from the initialization vector in step 3 by using a maximum pooling method as a feature extraction method of a first pooling layer, and multiple feature values X₃ form a new vector group X₄. The feature vectors may be expressed using the following formulas.

X₃={α₁,α₂, . . . ,αₖ}

X₄=max(X₃)

In step 5, a new feature vector X₆ is obtained by processing the feature X₄ obtained in step 4 through two convolutional layers again, wherein the function ELU( ) is selected as activation functions of the convolutional layers, weight vectors of neurons are Wₖ⁽⁵⁾ and Wₖ⁽⁶⁾ and a uniform distribution is applied to Wₖ⁽⁵⁾ and Wₖ⁽⁶⁾ to obtain initialization vectors.

In step 6, a new feature value X₆ is re-extracted from the initialization vector obtained in step 5 by using a maximum pooling method as a feature extraction method of a second pooling layer, and multiple feature values X₆ form a new vector group X₇. The feature vectors may be expressed using the following formulas.

X₆={α₆₁,α₆₂, . . . α₆ₖ}

X₇=max(X₆)

In step 7, a new feature vector X₁₁ is obtained by processing the feature X₇ obtained in step 6 through four convolutional layers again, wherein the function ELU( ) is selected as activation functions of the convolutional layers, weight vectors of neurons are Wₖ⁽⁷⁾, Wₖ⁽⁸⁾, Wₖ⁽⁹⁾ and Wₖ⁽¹⁰⁾, and a uniform distribution is applied to Wₖ⁽⁷⁾, Wₖ⁽⁸⁾, Wₖ⁽⁹⁾ and Wₖ⁽¹⁰⁾ to obtain initialization vectors.

In step 8, a new feature value X₁₁ is re-extracted from the initialization vector in step 7 by using a maximum pooling method as a feature extraction method of a third pooling layer, and multiple feature values X₁₁ form a new vector group X₁₂. The feature vectors may be expressed using the following formulas.

X₁₁={α₁₁₁,α₁₁₂, . . . ,α₁₁ₖ}

X₁₂=max(X₁₁)

In step 9, a new feature vector X₁₆ is obtained by processing the feature X₁₂ obtained in step 8 through four convolutional layers again, wherein the function ELU( ) is selected as activation functions of the convolutional layers, weight vectors of neurons are Wₖ⁽¹¹⁾, Wₖ⁽¹²⁾, Wₖ⁽¹³⁾ and Wₖ⁽¹⁴⁾, and a uniform distribution is applied to Wₖ⁽¹¹⁾, Wₖ⁽¹²⁾, Wₖ⁽¹³⁾ and Wₖ⁽¹⁴⁾ to obtain initialization vectors.

In step 10, a new feature value X₁₆ is re-extracted from the initialization vector in step 9 by using a maximum pooling method as a feature extraction method of a fourth pooling layer, and multiple feature values X₁₆ form a new vector group X₁₇. The feature vectors may be expressed using the following formulas.

X₁₆={α₁₆₁,α₁₆₂, . . . ,α₁₆ₖ}

X₁₇=max(X₁₆)

In step 10, a 200-dimensional feature vector is obtained by processing the convolutional layer feature vector X₁₇ obtained by steps 2-9 through a fully connected layer. Here, a uniform distribution is applied to Wₖ⁽¹⁷⁾ to obtain an initial vector value, ŷₖϵ{0,1,2,4,5,6,7} outputs eight-class emotion recognition, and the following function softplus( ) is used as an activation function. With the following formula:

ŷₖ=Wₖ⁽¹¹⁾*X̅+bₖ⁽¹¹⁾

softplus(ŷₖ)=log(1+e^{ŷ}^{ₖ})

an eight-class expression recognition model of the convolutional neural network is constructed.

In step 11, parameter values of the neural network are denoted using a stochastic gradient descent method as:

Wₖ⁽¹⁾, Wₖ⁽²⁾, Wₖ⁽³⁾, Wₖ⁽⁴⁾, Wₖ⁽⁵⁾, Wₖ⁽⁶⁾, Wₖ⁽⁷⁾, Wₖ⁽⁸⁾, Wₖ⁽⁹⁾, Wₖ⁽¹⁰⁾, Wₖ⁽¹¹⁾, Wₖ⁽¹²⁾, Wₖ⁽¹³⁾, Wₖ⁽¹⁴⁾, Wₖ⁽¹⁵⁾, Wₖ⁽¹⁶⁾, bₖ⁽¹⁾, bₖ⁽²⁾, bₖ⁽³⁾, bₖ⁽⁴⁾, bₖ⁽⁵⁾, bₖ⁽⁶⁾, bₖ⁽⁷⁾, bₖ⁽⁸⁾, bₖ⁽⁹⁾, bₖ⁽¹⁰⁾, bₖ⁽¹¹⁾, bₖ⁽¹²⁾, bₖ⁽¹³⁾, bₖ⁽¹⁴⁾, bₖ⁽¹⁵⁾, bₖ⁽¹⁶⁾.

In step 12, a weight for mapping X to an input vector y is denoted as θⱼ, a partial derivative of θⱼ is calculated to obtain a gradient at this time, and θⱼ is adjusted to minimize a loss function of j(θⱼ). Here, a learning rate determines a rate at which the parameter θⱼ moves to an optimal value, and a momentum factor controls a degree of influence of previous update of a weight on current update of a weight.

In step 13, the training is completed, and parameter values of the 8-class expression recognition model based on the convolutional neural network are obtained as:

Wₖ⁽¹⁾, Wₖ⁽²⁾, Wₖ⁽³⁾, Wₖ⁽⁴⁾, Wₖ⁽⁵⁾, Wₖ⁽⁶⁾, Wₖ⁽⁷⁾, Wₖ⁽⁸⁾, Wₖ⁽⁹⁾, Wₖ⁽¹⁰⁾, Wₖ⁽¹¹⁾, Wₖ⁽¹²⁾, Wₖ⁽¹³⁾, Wₖ⁽¹⁴⁾, Wₖ⁽¹⁵⁾, Wₖ⁽¹⁶⁾, bₖ⁽¹⁾, bₖ⁽²⁾, bₖ⁽³⁾, bₖ⁽⁴⁾, bₖ⁽⁵⁾, bₖ⁽⁶⁾, bₖ⁽⁷⁾, bₖ⁽⁸⁾, bₖ⁽⁹⁾, bₖ⁽¹⁰⁾, bₖ⁽¹¹⁾, bₖ⁽¹²⁾, bₖ⁽¹³⁾, bₖ⁽¹⁴⁾, bₖ⁽¹⁵⁾, bₖ⁽¹⁶⁾.

In step S2022, an SVM algorithm is applied to the obtained emotion recognition feature vector to determine an expression of the target object. For example, with the above step S2021, the emotion recognition feature vector is obtained through the emotion recognition model, and in this step, eight expressions are recognized using the SVM algorithm. For example, the eight expressions comprise neuter, happiness, surprise, sadness, anger, disgust, fear or contempt. In this step, the expression of the target object may be recognized as one of the above eight expressions.

FIG. 4b illustrates a flowchart of an example of step S203 of FIG. 3.

In step S2031, the acceleration information is compared with reference information.

In step S2032, a behavioral performance of the target object is determined according to a comparison result. For example, the behavioral performance may comprise one of “tension”, “calmness”, or “negativity”.

In step S2033, a real-time emotional state of the target object is determined by combining the determined expression with the determined behavioral performance. For example, the behavioral performance of “calmness” and the expression of “happiness” may be combined into a real-time emotional state of “calm happiness”. Of course, the embodiments of the present disclosure are not limited thereto, and any other combination manner may be used as needed to obtain the real-time emotional state of the target object. For example, a comprehensive emotion state etc. is obtained by performing weighted calculation on a degree of “calmness” and a degree of “happiness”.

The emotion analysis method and device described above with reference to FIG. 2 to FIG. 4 may be applied to mobile portable devices such as mobile phones, tablet computers, and customer service systems etc.

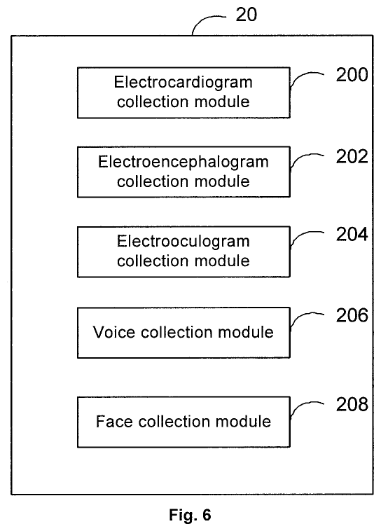

The embodiments of the emotion analysis device and method in a case where the body parameter information comprises the physiological information of the target object will be described below with reference to FIGS. 5 to 8.

In a third aspect, the present disclosure proposes another emotion analysis device, and FIG. 5 illustrates a schematic block diagram of an emotion analysis device according to another embodiment of the present disclosure. In the present embodiment, the body parameter information comprises physiological information of the target object. As shown in FIG. 5, the emotion analysis device 2 comprises: a multi-dimensional information collection module 20 configured to collect a facial image, acceleration information, and various physiological information of the target object; an emotional stress analysis module 22 configured to recognize an expression and an identity of the target object according to the facial image, and comprehensively analyzes an emotional stress state of the target object in combination with the acceleration information and various physiological information; and a result presentation module 24 configured to present the emotional stress state of the target object.

In the emotion analysis device according to the embodiment of the present disclosure, collection and analysis of the facial image, the acceleration information, and various physiological information are integrated, and the emotional health state of the target object is comprehensively analyzed and determined, which overcomes misjudgment caused by emotion recognition according to a single factor, so that the emotional health analysis and management are more comprehensive and accurate.

In some embodiments, as shown in FIG. 6, the multi-dimensional information collection module 20 may comprise: an electrocardiogram collection module 200, an electroencephalogram collection module 202, an electrooculogram collection module 204, a voice collection module 206, and a face collection module 208, which are configured to collect electrocardiogram signal data, electroencephalogram signal data, electrooculogram signal data, voice signal data, facial image data and facial expression data respectively.

In some embodiments, the emotional stress analysis module 22 may comprise an electrocardiogram-electroencephalogram-electrooculogram emotion analysis module, a voice emotion analysis module, and a facial expression analysis module. Here, the electrocardiogram-electroencephalogram-electrooculogram emotion analysis module comprises: an electrocardiogram analysis module, a heart rate variability analysis module, an electroencephalogram analysis module, and an electrooculogram analysis module.

The electrocardiogram analysis module is configured to analyze an electrocardiogram signal, and obtain feature points of the electrocardiogram signal, comprising a R point, a QRS wave group, a Q point, an S point, a P wave, an ST wave band, and a T wave of the electrocardiogram signal, through analysis using a P-T algorithm.

The heart rate variability analysis module is configured to obtain real-time heart rate variability using a heart rate frequency domain analysis method. The heart rate variability analysis module comprises a heart rate variability emotion analysis module which recognizes different emotional states by analyzing HF, LF, and HF/LF of the heart rate variability in different emotional states. For example, in a tense state, a ratio of HF/LF of the heart rate variability decreases; in a happy emotion, the HF power of the heart rate variability increases, and in a sad emotion, the HF power of the heart rate variability decreases; and in a sad state, the LF power increases, and in a happy state, the LF power decreases. The heart rate variability emotion analysis module analyzes different emotional states according to parameters of the heart rate variability as a factor for analyzing different emotional states.

The electroencephalogram analysis module obtains features of the electroencephalogram signal by analyzing an alpha rhythm, a Beta rhythm, and a Theta rhythm of a brain wave. The electroencephalogram analysis module further comprises a brain wave emotion analysis module, which extracts a brain wave signal using LZ complexity and an approximate entropy parameter method, and performs emotion reorganization by applying the extracted alpha rhythm, Beta rhythm and ApEn+LLE feature of the brain wave in different states to an algorithm such as SVM etc.

The electrooculogram analysis module obtains emotional states of the monitored person by analyzing an electrooculographic behavior trajectory in different emotional states.

The electrocardiogram-electroencephalogram-electrooculogram emotion analysis module comprehensively analyzes an emotion of the target object based on analysis results of the electrocardiogram analysis, the electroencephalogram analysis, and the electrooculogram analysis. The voice emotion analysis module analyzes the emotion of the target object based on a result of the voice analysis, and the facial expression analysis module analyzes the emotion of the target object based on a result of the facial expression recognition.

In some embodiments, the electrocardiogram-electroencephalogram-electrooculogram emotion analysis module may be configured to acquire the emotion and a psychological stress state of the target object by inputting the LF, the HF, the HF/LF, the alpha rhythm, the Beta rhythm, the ApEn+LLE feature and the electrooculographic behavior trajectory of the heart rate variability of the electrocardiogram signal analyzed by various analysis modules described above into Decision Tables and Naive Bayes (DTNB). For example, the emotion of the target object comprises “positivity”, “neuter” and “negativity”, and the psychological stress state of the target object comprises ‘too low stress’, “normal stress” and “excessive stress”. An exemplary operation process of the electrocardiogram-electroencephalogram-electrooculogram emotion analysis module is as follows.

In step 1, the measured values of the LF, the HF, the HF/LF, the alpha rhythm, the Beta rhythm, and the ApEn+LLE are normalized respectively.

In step 2, the above normalized data is input into the DTNB for training to obtain a model A suitable for making a decision.

In step 3, the re-measured values of the LF, the HF, the HF/LF, the alpha rhythm, the Beta rhythm, and the ApEn+LLE are normalized.

In step 4, the normalized data is input into the model A, and a corresponding emotional state is determined by the model A.

In some embodiments, the facial expression analysis module comprises a face detection module, a face recognition module, and an emotion recognition module.

The face detection module performs face detection using a face detection algorithm, such as a color image fast face detection algorithm based on wavelet transformation. Specific steps are as follows.

In step 1, nonlinear transformation is performed on the facial image.

In step 2, high-frequency components of a face are extracted by using wavelet transformation.

In step 3, a hidden layer function of a multi-layer neural network is replaced with a wavelet kernel function.

In step 4, face detection is performed using the multi-layer neural network.

The face recognition module performs face recognition using a face recognition algorithm, such as a Local Binary Patterns (LBP) algorithm. Specific steps are as follows.

In step 1, an original facial image is partitioned.

In step 2, a local difference value and a center pixel gray value of each partitioned image are analyzed.

In step 3, histogram statistical features of each partitioned image are extracted using calculation operators S_{LBP}^{W}^{₂}(P,R) and M_{LBP}^{W}^{₂}(P,R) of a bilinear interpolation algorithm respectively, wherein S and M represent a x-coordinate and a y-coordinate of a point obtained using bilinear interpolation respectively, P represents a number of sampling points around the point, R represents a radius of a circle, and μ₂ represents the point (i.e., a center point).

In step 4, a sequence of LBP histograms of all the partitioned images are connected to obtain LBP features of the facial image, which are used as features for face recognition.

In step 5, a dissimilarity between the histograms is calculated using a Chi square statistical method, and the histograms are classified using a nearest neighbor criterion.

The emotion recognition module performs emotion recognition using an emotion recognition algorithm, such as a CNN algorithm. Specific steps are as follows.

In step 1, the facial expression image is pre-processed by normalization.

In step 2, implicit features are extracted using a trainable convolution kernel.

In step 3, dimensionality of the extracted implicit features is reduced using a maximum pooling method.

In step 4, an expression of a test sample image is classified and recognized by a Softmax classifier.

The face detection, face recognition, and emotion recognition described above may also be applied to the recognition of the expression and the identity in the embodiments described above.

According to a specific embodiment of the present disclosure, in the emotion analysis device, the emotion analysis module inputs the emotional psychological stress state results analyzed according to the electrocardiogram-electroencephalogram-electrooculogram emotion analysis module, the facial expression analysis module, and the voice emotion analysis module to a Bayesian network, to obtain a comprehensive evaluation result. A process is as follows.

i. Collected data is divided into a training set and a test set.

ii. Emotional-psychological stress states of “positivity-low psychological stress”, “neuter-normal psychological stress”, and “negativity-high psychological stress” are set to “1”, “2”, “3” respectively.

iii. The Bayesian network of the emotional-psychological stress is trained with more than 10 repeated iterations.

iv. Least error parameters of the Bayesian network are obtained:

$$p ( H \vert E , … , {E}_{i} ) = \frac{p ( H ) p ( E , … , {E}_{i} \vert H )}{p ( E )}$$

where H is a hypothesis variable; E is an evidence variable, and p(H|E, . . . , Eᵢ) is a probability that an emotion of H occurs under a condition that conditions of E, . . . , Eᵢ are satisfied at the same time.

In a fourth aspect, according to the above embodiment, the present disclosure further provides an emotion analysis method. FIG. 7 illustrates a flowchart of an emotion analysis method according to an embodiment of the present disclosure. In the present embodiment, the body parameter information comprises physiological information of the target object. As shown in FIG. 7, the emotion analysis method comprises the following steps.

In step S301, a facial image and physiological information of the target object are collected.

In step S302, an expression of the target object is recognized according to the facial image, and an emotional stress state of the target object is determined according to the expression in combination with the physiological information.

In some embodiments, the emotion analysis method may further comprise presenting the emotional stress state of the target object.

In some embodiments, the emotion analysis method may further comprise recognizing an identity of the target object according to the collected facial image.

In some embodiments, the emotion analysis method may further comprise collecting acceleration of the target object and comprehensively considering the expression, the physiological information, and the acceleration information when the emotional stress state of the target object is determined. For example, the behavioral performance of the target object may be determined according to the acceleration information, so as to determine an emotion of the target object in behavioral performance, an emotion of the target object in physiology is determined according to the physiological information, an emotion of the target object in facial expression is determined according to the expression of the target object, and an emotional stress state of the target object is determined by comprehensively considering the above emotions in various aspects.

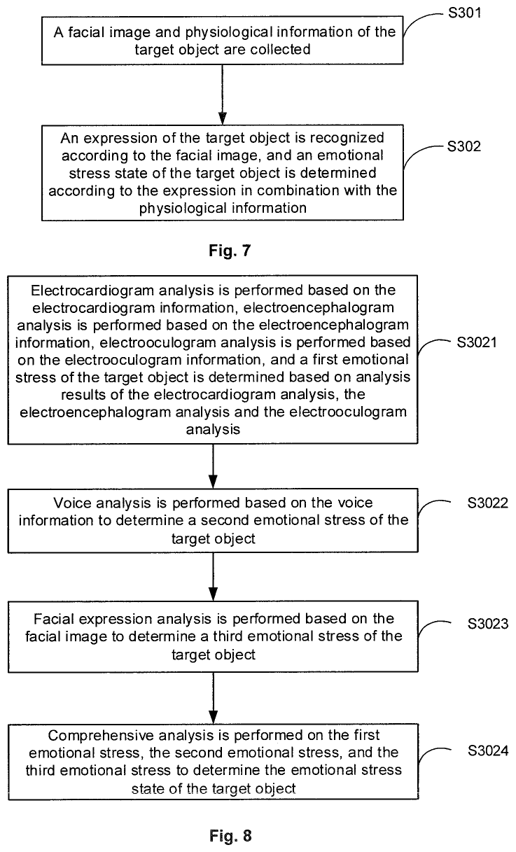

FIG. 8 illustrates a flowchart of an example of step S302 of FIG. 7.

In step S3021, electrocardiogram analysis is performed based on the electrocardiogram information, electroencephalogram analysis is performed based on the electroencephalogram information, electrooculogram analysis is performed based on the electrooculogram information, and a first emotional stress of the target object is determined based on analysis results of the electrocardiogram analysis, the electroencephalogram analysis and the electrooculogram analysis. For example, a Decision Tables and Naive Bayes (DTNB) algorithm is applied using at least one of a low frequency power LF, a high frequency power HF, and a high frequency power to low frequency power ratio HF/LF of heart rate variability obtained by the electrocardiogram analysis, at least one of an alpha rhythm, a Beta rhythm, and an ApEn+LLE feature obtained by the electroencephalogram analysis, and an electrooculographic behavior trajectory determined by the electrooculogram analysis as inputs to obtain the first emotional stress of the target object.

In step S3022, voice analysis is performed based on the voice information to determine a second emotional stress of the target object.

In step S3023, facial expression analysis is performed based on the facial image to determine a third emotional stress of the target object. The facial expression analysis may comprise face detection, face recognition, and emotion recognition.

In step S3024, comprehensive analysis is performed on the first emotional stress, the second emotional stress, and the third emotional stress to determine the emotional stress state of the target object. For example, the first emotional stress, the second emotional stress, and the third emotional stress are input to a Bayesian network to obtain a comprehensive evaluation result as the emotional stress state of the target object.

The emotion analysis method and device described above with reference to FIGS. 5 to 7 may be applied in a medical system having a physiological information detection device (for example, an electrocardiogram detector, an electroencephalogram detector, an electrooculogram detector). For example, the emotion analysis method and device described above may be implemented in a computer or server for medical data analysis in a hospital, or may also be implemented in a computer or server dedicated to emotion analysis.

The embodiments of the present disclosure further provide an emotion analysis device comprising a memory and a processor, the memory having stored thereon instructions which, when executed by the processor, cause the processor to perform any of the emotion analysis method according to any of the above embodiments.

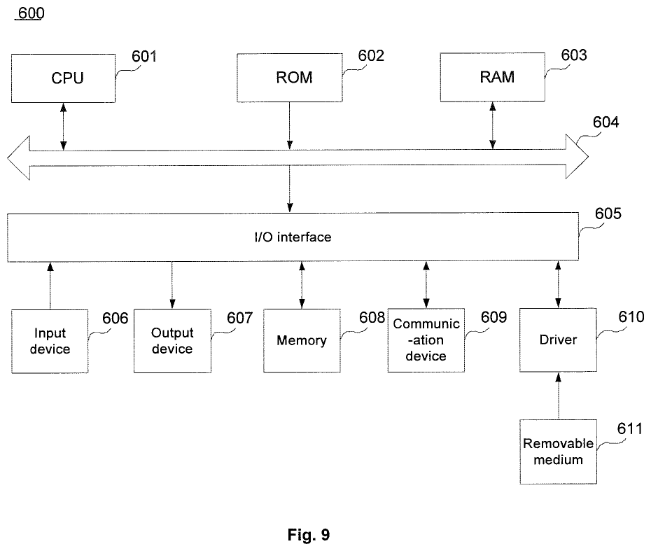

FIG. 9 illustrates a schematic diagram of a computer device 600 in which the above emotion analysis method may be implemented, according to an embodiment of the present disclosure. As shown in FIG. 9, the computer device 600 comprises a Central Processing Unit (CPU) 601 which may perform various appropriate actions and process the emotion analysis method according to any of the above embodiments based on a program stored in a Read Only Memory (ROM) 602 or a program loaded from a storage portion into a Random Access Memory (RAM) 603. Various programs and data required for operations of a system are also stored in the RAM 603. The CPU 601, the ROM 602, and the RAM 603 are connected to each other through a bus 604. An Input/Output (I/O) interface 605 is also connected to the bus 604.

The following components are connected to the I/O interface 605: an input device 606 comprising a keyboard, a mouse, etc.; an output device 607 comprising a Cathode Ray Tube (CRT), a Liquid Crystal Display (LCD), etc., and a speaker; a memory 608 comprising a hard disk etc.; and a communication device 609 comprising a network interface card such as a LAN card, a modem, etc. The communication device 609 performs communication processing via a network such as the Internet. The driver is also connected to the I/O interface 605 as needed. A removable medium 611, such as a magnetic disk, an optical disk, a magneto-optical disk, a semiconductor memory etc., is mounted on the driver 610 as needed, so that a computer program read therefrom is installed into the memory 608 as needed.

In some embodiments, the emotion analysis method according to any of the above embodiments may be implemented as a computer software program. For example, the embodiments of the present disclosure provide a computer program product comprising a computer program carried on a computer readable medium, the computer program comprising program codes for performing the methods illustrated in the above flowcharts. In such an embodiment, the computer program may be downloaded and installed from the network via a communication portion, and/or installed from a removable medium. When the computer program is executed by the CPU 601, the computer program causes the CPU 601 to execute the emotion analysis method according to any of the above embodiments.

The embodiments of the present disclosure further provide a computer readable storage medium having stored thereon computer readable instructions which, when executed by a computer, cause the computer to perform the emotion analysis method according to any of the above embodiments.

It should be illustrated that the computer readable medium shown in the present disclosure may be a computer readable signal medium or a computer readable storage medium or any combination thereof. The computer readable storage medium may be, for example, but not limited to, an electronic, magnetic, optical, electromagnetic, infrared, or semiconductor system, apparatus, or device, or any combination thereof. More specific examples of computer readable storage media may comprise, but not limited to, an electrical connection having one or more wires, a portable computer disk, a hard disk, a Random Access Memory (RAM), a Read Only Memory (ROM), an Erasable Programmable Read Only Memory (EPROM or flash memory), an optical fiber, a portable Compact Disk Read Only Memory (CD-ROM), an optical storage device, a magnetic storage device, or any suitable combination thereof. In the present disclosure, the computer readable storage medium may be any tangible medium which may contain or store a program, which may be used by or in connection with an instruction execution system, apparatus, or device. In the present disclosure, the computer readable signal medium may comprise a data signal which is propagated in a baseband or as a part of a carrier, and carries computer readable program codes. The propagated data signal may have a variety of forms comprising, but not limited to, an electromagnetic signal, an optical signal, or any suitable combination thereof. The computer readable signal medium may also be any computer readable medium other than the computer readable storage medium, which may transmit, propagate, or transport a program for use by or in connection with the instruction execution system, apparatus, or device. The program codes contained in the computer readable medium may be transmitted by any suitable medium, comprising, but not limited to, a wireless connection, a wire, a fiber, an optic cable, RF, etc., or any suitable combination thereof.

The flowcharts and block diagrams in the accompanying drawings illustrate architecture, functions, and operations of possible implementations of systems, methods, and computer program products according to various embodiments of the present disclosure. In this regard, each block of the flowcharts or block diagrams may represent a module, a program segment, or a portion of codes, which comprises one or more executable instructions for implementing specified logic functions. It should also be illustrated that in some alternative implementations, the functions illustrated in the blocks may also occur in a different order than that illustrated in the accompanying drawings. For example, two successively represented blocks may in fact be executed substantially in parallel, or they may sometimes be executed in a reverse order, depending upon functionality involved. It is also illustrated that each block of the block diagrams or flowcharts, and combinations of blocks in the block diagrams or flowcharts, may be implemented by a dedicated hardware-based system which performs specified functions or operations, or may be implemented by combination of dedicated hardware and computer instructions.

Units described in the embodiments of the present disclosure may be implemented by software, or may be implemented by hardware, and the described units may also be disposed in a processor. Here, names of these units do not in any way constitute a limitation on the units themselves. The described units or modules may also be disposed in a processor.

In another aspect, the computer readable medium according to the embodiment of the present disclosure may also be included in the electronic device described in the above embodiment; or may exist separately but is not assembled into the electronic device. The above computer readable medium carries one or more programs which, when executed by one of the electronic devices, cause the electronic device to implement the emotion analysis method as described in the above embodiments. The above computer readable medium carries one or more programs which, when executed by one of the electronic devices, cause the electronic device to implement the emotion analysis method as described in the above embodiments.

It should be illustrated that although several modules or units of a device for performing an action are mentioned in the detailed description above, such division is not mandatory. In fact, according to the embodiments of the present disclosure, features and functions of two or more modules or units described above may be embodied in one module or unit. Conversely, features and functions of one of the modules or units described above may be further divided to be embodied by multiple modules or units.

In addition, although various steps of the method according to the present disclosure are described in a specific order in the accompanying drawings, it is not required or implied that the steps must be performed in the specific order, or all the steps shown must be performed to achieve a desired result. Additionally or alternatively, certain steps may be omitted, multiple steps may be combined into one step to be executed, and/or one step may be decomposed into multiple steps to be executed etc.

Through the description of the above embodiments, those skilled in the art will readily understand that exemplary embodiments described herein may be implemented by software or by software in combination with necessary hardware.

It should be illustrated that the above embodiments are only for explaining the technical solutions according to the present disclosure, and are not limited thereto. Although the present disclosure has been described in detail with reference to the above embodiments, those skilled in the art should understand that they may still modify the technical solutions described in the above embodiments, or make equivalent substitutions to some of the technical features. These modifications and substitutions do not cause the essence of the corresponding technical solutions to depart from the spirit and scope of the technical solutions according to the embodiments of the present disclosure.

I/We claim:

1. An emotion analysis method, comprising:
collecting a facial image and body parameter information of a target object; and
recognizing an expression of the target object according to the facial image, and determining an emotional state of the target object according to the recognized expression in combination with the body parameter information.

2. The emotion analysis method according to claim 1, wherein the body parameter information comprises acceleration information of the target object.

3. The emotion analysis method according to claim 2, wherein recognizing an expression of the target object according to the facial image comprises:
applying a deep convolutional neural network algorithm to the facial image to obtain an emotion recognition feature vector; and
applying a Support Vector Machine (SVM) algorithm to the obtained emotion recognition feature vector to determine the expression of the target object.

4. The emotion analysis method according to claim 3, wherein the expression comprises one of neuter, happiness, surprise, sadness, anger, disgust, fear or contempt.

5. The emotion analysis method according to claim 2, wherein determining an emotional state of the target object according to the recognized expression in combination with the body parameter information comprises:
comparing the acceleration information with reference information;
determining a behavioral performance of the target object according to a comparison result; and
determining a real-time emotional state of the target object by combining the determined expression with the determined behavioral performance.

6. The emotion analysis method according to claim 5, wherein the behavioral performance comprises one of “tension”, “calmness” or “negativity”.

7. The emotion analysis method according to claim 2, further comprising: determining whether the target object has an abnormal emotion according to the obtained emotional state, and if so, issuing a reminder.

8. The emotion analysis method according to claim 2, further comprising: collecting fingerprint information of the target object, and recognizing an identity of the target object according to the collected facial image or fingerprint information.

9. The emotion analysis method according to claim 1, wherein the body parameter information comprises physiological information of the target object.

10. The emotion analysis method according to claim 9, wherein the physiological information comprises: electrocardiogram information, electroencephalogram information, electrooculogram information, and voice information.

11. The emotion analysis method according to claim 10, wherein recognizing an expression of the target object according to the facial image, and determining an emotional state of the target object according to the recognized expression in combination with the body parameter information comprises:
performing electrocardiogram analysis based on the electrocardiogram information, performing electroencephalogram analysis based on the electroencephalogram information, performing electrooculogram analysis based on the electrooculogram information, and determining a first emotional stress of the target object based on analysis results of the electrocardiogram analysis, the electroencephalogram analysis and the electrooculogram analysis;
performing voice analysis based on the voice information to determine a second emotional stress of the target object;
performing facial expression analysis based on the facial image to determine a third emotional stress of the target object; and
performing comprehensive analysis on the first emotional stress, the second emotional stress, and the third emotional stress to determine an emotional stress state of the target object.

12. The emotion analysis method according to claim 11, wherein determining a first emotional stress of the target object based on analysis results of the electrocardiogram analysis, the electroencephalogram analysis and the electrooculogram analysis comprises:
applying a Decision Tables and Naive Bayes (DTNB) algorithm using at least one of a low frequency power LF, a high frequency power HF, and a high frequency power to low frequency power ratio HF/LF of heart rate variability obtained by the electrocardiogram analysis, at least one of an alpha rhythm, a Beta rhythm, and an ApEn+LLE feature obtained by the electroencephalogram analysis, and an electrooculographic behavior trajectory determined by the electrooculogram analysis as inputs to obtain the first emotional stress of the target object.

13. The emotion analysis method according to claim 11, wherein the facial expression analysis comprises face detection, face recognition, and emotion recognition.

14. The emotion analysis method according to claim 11, wherein performing comprehensive analysis on the first emotional stress, the second emotional stress, and the third emotional stress to determine the emotional stress state of the target object comprises: inputting the first emotional stress, the second emotional stress, and the third emotional stress to a Bayesian network to obtain a comprehensive evaluation result as the emotional stress state of the target object.

15. The emotion analysis method according to claim 9, further comprising: recognizing an identity of the target object according to the collected facial image.

16. The emotion analysis method according to claim 1, further comprising: presenting the emotional state of the target object.

17. An emotion analysis device, comprising a memory and a processor, the memory having stored thereon instructions which, when executed by the processor, cause the processor to perform the emotion analysis method according to claim 1.

18. A computer readable storage medium having stored thereon computer readable instructions which, when executed by a computer, cause the computer to perform the emotion analysis method according to claim 1.
